(19) United States
(12) Patent Application Publication
(10) Pub. No.: US 2014/0194954 A1
(43) Pub. Date: Jul. 10, 2014

(54) PHOTORADIATION DEVICE AND PHOTORADIATION THERAPY/PROPHYLAXIS DEVICE COMPRISING SAME

(71) Applicant: Kazuhiro Daijo, Osaka (JP)

(72) Inventor: Kazuhiro Daijo, Osaka (JP)

(73) Assignee: PANASONIC CORPORATION, Kadoma-shi, Osaka (JP)

(21) Appl. No.: 14/238,505

(22) PCT Filed: Aug. 22, 2012
(86) PCT No.: PCT/JP2012/005243

§ 371 Date: Feb. 12, 2014

(30) Foreign Application Priority Data
Sep. 2, 2011 (JP) 2011-191348

(51) Int. Cl. A61N 5/06

(52) U.S. Cl. CPC A61N 5/06
USPC 607/88; 250/492.1

(57) ABSTRACT

A photoradiation device of the present invention includes a light emitting unit that radiates radiation light; a reflection unit that reflects the radiation light; and a light guide unit that guides the reflected radiation light to an irradiation target body. The light guide unit includes a first light guide face and a second light guide face. The reflection unit includes a first reflection unit that reflects a part of the radiation light to the first light guide face, a second reflection unit that reflects entering light to the second light guide face, and a third reflection unit formed of a main body part provided with a transmission unit which reflects a part of remainder of the radiation light to the second reflection unit as the entering light. The third reflection unit is disposed with a transmission unit side inclined toward a light emitting unit such that the shorter a distance of the transmission unit to the light emitting unit, the larger an amount of transmission light of the radiation light. Thus, a photoradiation device that irradiates a plurality of irradiated faces with a single light emitting unit is achieved.

This application is a U.S. National Phase application of PCT International Application PCT/JP2012/005243.

### TECHNICAL FIELD

The present invention relates to a photoradiation device capable of uniformly irradiating an irradiation target body with light and a photoradiation therapy/prophylaxis device including the photoradiation device.

### BACKGROUND ART

Conventionally, as a photoradiation device for irradiating an irradiation target body, various devices have been proposed. Examples thereof include a photoradiation device provided with a light guide member for planarly irradiating irradiation target bodies which are disposed uniformly and spread planarly.

Furthermore, as a photoradiation device provided with an irradiation member, a lighting apparatus for a vending machine has been proposed according to demands for energy-saving, space-saving, and miniaturization, or the like (see, for example, Patent Literature 1).

The lighting apparatus for a vending machine disclosed in Patent Literature 1 includes a flat diffusion panel disposed in adjacent to a plurality of vertically and horizontally arranged commodity samples, a flat reflection sheet disposed facing an opposite side (rear side) to the commodity samples of the diffusion panel, and a light emitting unit disposed on both one lateral sides of the diffusion panel and the reflection sheet. The reflection sheet is disposed inclined toward the diffusion panel in such a manner that the distance to the diffusion panel is shortened (narrowed) as the reflection sheet is apart from the light emitting unit.

Then, the lighting apparatus for a vending machine allows radiation light radiated from the light emitting unit to be reflected by the reflection sheet and to enter the diffusion panel. At this time, as a distance from the light emitting unit is increased, a (clearance) distance from the reflection sheet to the diffusion panel is shortened. That is to say, as an optical path length of each radiation light from the light emitting unit to the reflection sheet is increased, an optical path length of each radiation light from the reflection sheet to the diffusion panel is shortened. Therefore, the reflection sheet is disposed such that a distance from the light emitting unit to the reflection sheet and a distance from the reflection sheet to the diffusion panel are the same as each other, and the optical path length of each radiation light reflected by the reflection sheet and reaching the diffusion panel from light emitting unit becomes uniform.

That is to say, a conventional lighting apparatus for a vending machine is designed such that illumination intensity on the diffusion panel becomes uniform by adjusting the optical path length of radiation light radiated from the light emitting unit to the reflection sheet in order not to lower the illumination intensity of the radiation light irradiated from the diffusion panel even if a distance from the light emitting unit is increased.

However, the above-mentioned lighting apparatus for a vending machine corresponds to irradiation of an irradiation target body from only one side. Therefore, for example, when the irradiation target body is a hand, in order to irradiate both the palm and the back of a hand with light at the same time, a lighting apparatus for a vending machine must be disposed facing each of the both sides of the hand.

In the above-mentioned configuration, there have been problems including increase of the number of components and increase of consumed energy. Therefore, a photoradiation device capable of uniformly irradiating a plurality of irradiated faces such as both sides of the hand by a single light emitting unit has been strongly demanded.

### CITATION LIST

### Patent Literature

PTL 1: Japanese Patent Unexamined Publication No. 2009-282725

### SUMMARY OF THE INVENTION

In order to solve the above-mentioned problems, a photoradiation device of the present invention includes a light emitting unit that radiates radiation light; a reflection unit that reflects the radiation light; and a light guide unit that guides the radiation light reflected from the reflection unit to an irradiation target body. The light guide unit includes a first light guide face and a second light guide face. The reflection unit includes a first reflection unit that reflects a part of the radiation light to the first light guide face, a second reflection unit that reflects entering light to the second light guide face, and a third reflection unit formed of a main body part provided with a transmission unit which reflects a part of remainder of the radiation light to the second reflection unit as the entering light. Furthermore, the third reflection unit is disposed with a transmission unit side inclined toward a light emitting unit such that the shorter a distance of the transmission unit to the light emitting unit the larger an amount of transmission light of the radiation light.

Thus, the radiation light radiated from one light emitting unit is equally distributed to the first light guide face and the second light guide face. That is to say, the radiation light can be radiated to a plurality of light guide faces by one light emitting unit, and thereby an irradiation target body such as a hand having both front and rear sides can be irradiated simultaneously. Furthermore, it is possible to uniformly adjust illumination intensity distribution in the first light guide face by the transmission unit of the third reflection unit.

Furthermore, the present invention is a photoradiation therapy/prophylaxis device for carrying out treatment or prevention by irradiating a specific site of a living body with radiation light radiated from a photoradiation device, further including a wavelength transmitting unit which is disposed on an optical path of the radiation light radiated from the light emitting unit through the light guide unit, and allows radiation light in a wavelength range of not less than 566.5 nm and not more than 780 nm in the radiation light radiated from the light emitting unit. A specific site of a living body is irradiated with the radiation light which is allowed to transmit through the wavelength transmitting unit.

Thus, production of inflammatory cytokine can be inhibited so as to prevent affection of, for example, an inflammatory disease and to reduce or inhibit symptoms at the time of affection of the disease.

### BRIEF DESCRIPTION OF DRAWINGS

FIG. 1A is a graph showing a production amount of a vascular endothelial cell growth factor (hVEGF) for each wavelength in accordance with an exemplary embodiment of the present invention.

FIG. 1B is a graph showing a production ratio of inflammatory cytokine for each wavelength in accordance with the exemplary embodiment of the present invention.

FIG. 2 is a control circuit diagram of a photoradiation therapy/prophylaxis device in accordance with the exemplary embodiment of the present invention.

FIG. 3 is an entire perspective view of the photoradiation therapy/prophylaxis device in accordance with this exemplary embodiment.

FIG. 4 is a sectional view of an optical system of the photoradiation therapy/prophylaxis device in accordance with this exemplary embodiment.

FIG. 5A is a front view showing a third reflection unit of the photoradiation therapy/prophylaxis device in accordance with this exemplary embodiment.

FIG. 5B is a partially enlarged sectional view showing arrangement of the third reflection unit of the photoradiation therapy/prophylaxis device in accordance with this exemplary embodiment.

FIG. 6 is a graph showing spectral characteristics of radiation light of a band-pass filter (a wavelength transmitting unit) used for the photoradiation therapy/prophylaxis device in accordance with this exemplary embodiment.

FIG. 7A is a distribution diagram of illumination intensity in an irradiation target surface of radiation light irradiated from a first light guide face of the irradiation target surface of the photoradiation therapy/prophylaxis device in accordance with Example of this exemplary embodiment.

FIG. 7B is a distribution diagram of illumination intensity in an irradiation target surface of radiation light irradiated from a second light guide face of the irradiation target surface of the photoradiation therapy/prophylaxis device in accordance with Example of this exemplary embodiment.

FIG. 8A is a distribution diagram of illumination intensity in an irradiation target surface of radiation light irradiated from a first light guide face of the irradiation target surface of the photoradiation therapy/prophylaxis device in accordance with a Comparative Example of the exemplary embodiment.

FIG. 8B is a distribution diagram of illumination intensity in an irradiation target surface of radiation light irradiated from a second light guide face of the irradiation target surface of the photoradiation therapy/prophylaxis device in accordance with a Comparative Example of the exemplary embodiment.

FIG. 9A is a graph showing a relation between a position of an irradiation target surface of the radiation light irradiated from the first light guide face and illumination intensity in the illumination intensity distribution diagram of the photoradiation therapy/prophylaxis device in Example of FIG. 7A and Comparative Example of FIG. 8A.

FIG. 9B is a graph showing a relation between a position of an irradiation target surface of the radiation light irradiated from the second light guide face and illumination intensity in the illumination intensity distribution diagram of the photoradiation therapy/prophylaxis device in Example of FIG. 7B and Comparative Example of FIG. 8B.

FIG. 10A is a front view showing the third reflection unit of another example of the photoradiation therapy/prophylaxis device in accordance with the exemplary embodiment of the present invention.

FIG. 10B is a front view showing the third reflection unit of still another example of the photoradiation therapy/prophylaxis device in accordance with the exemplary embodiment of the present invention.

### DESCRIPTION OF EMBODIMENTS

Hereinafter, a photoradiation device in accordance with an exemplary embodiment of the present invention and a photoradiation therapy/prophylaxis device having the same are described with reference to drawings. Note here that the present invention is not necessarily limited to this exemplary embodiment.

### Exemplary Embodiment

Hereinafter, a photoradiation device in accordance with exemplary embodiment of the present invention and a photoradiation therapy/prophylaxis device having the same are described with reference to drawings.

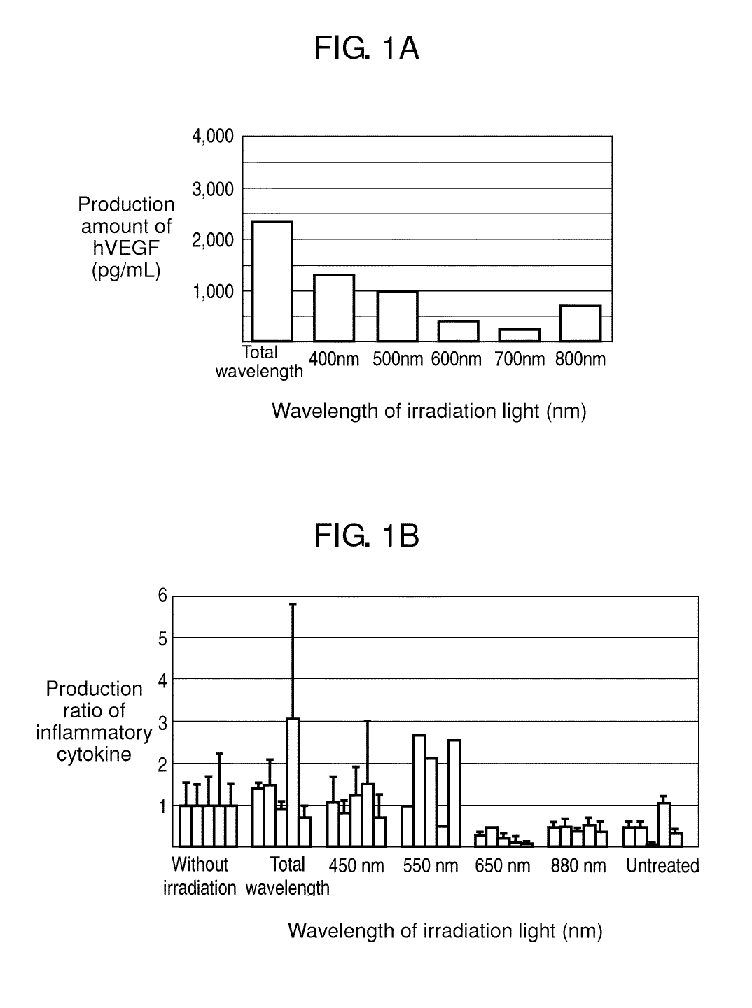

Firstly, an action for inhibiting production of inflammatory cytokines of a photoradiation therapy/prophylaxis device in this exemplary embodiment is described with reference to FIGS. 1 and 1B.

The inflammatory cytokines are one type of cytokines that are a generic name of soluble protein responsible for various intercellular information in a living body. In particular, the inflammatory cytokines are involved as a causative factor that causes various inflammation symptoms in a living body, and produced from activated macrophages or activated blood vessel endothelial cells.

Specific examples of the inflammatory cytokines include typical inflammatory cytokines verified in experiments and the like, that is, (h)VEGF (Vascular Endothelial Growth Factor), TNFα (tumor necrosis factor-α), IL-1β (interleukin-1β), IFNγ(interferonγ), IL-6 (interleukin-6), IL-12a (interleukin-12a), and the like.

The inflammatory cytokines exhibit directional activity as a whole while many types of cytokines form a complicated network in a living body. That is to say, the inflammatory cytokine is similarly produced from blood cells and elicits a disease state in which an inflammation reaction is excessive when a balance with respect to the anti-inflammatory cytokine having an activity of inhibiting inflammation is lost.

Note here that it has been proved from experiments or the like that IL-4 (interleukin 1α, interleukin-4) as one of the anti-inflammatory cytokines does not have an effect of inhibiting the production of inflammatory cytokines.

However, the applicant of the present application has found that the above-mentioned inflammatory cytokine inhibits a production amount of hVEGF at a specific wavelength of irradiation light (radiation light) irradiated from, for example, a discharge tube more strongly as compared with the other wavelength.

Specifically, as shown in FIG. 1 An irradiation light irradiated to a human epidermal cell by illuminating a xenon discharge tube is divided by a band-pass filter having a half width of 40 nm for each predetermined center wavelength, and production amounts of hVEGF for each center wavelength are compared with each other. As a result, it is shown that the production amount of hVEGF becomes minimum in the range from the center wavelength of 600 nm to the center wavelength of 700 nm.

Furthermore, as shown in FIG. 1B, similarly, irradiation light irradiated to a human epidermal cell by illuminating a xenon discharge tube is separated by the band-pass filter having a half width of 40 nm for each predetermined center wavelength, and production ratios of inflammatory cytokines for each center wavelength (a ratio with respect to a case in which irradiation is not carried out as a reference) are compared with each other. As a result, it is shown that the production ratio of the inflammatory cytokines becomes the lowest (strongly inhibited) in the center wavelength of 650 nm.

Note here that in FIG. 1B, the production ratio of the inflammatory cytokines for each wavelength of the irradiation light is shown by relative values by defining the production amount of inflammatory cytokine when the irradiation light is not irradiated as a reference (“1”). Furthermore, in FIG. 1B, for each wavelength (for example, 450 nm and 550 nm), the results of the production ratios of each inflammatory cytokine are shown sequentially in the orders of TNFα, IL-1β, IFNγ, IL-6, and IL-12a, from the left.

That is to say, from the above-mentioned results, production of inflammatory cytokine is inhibited by irradiating an affected area with irradiation light in appropriate wavelength, and thereby treatment of inflammatory disease, which has a new mechanism, can be carried out.

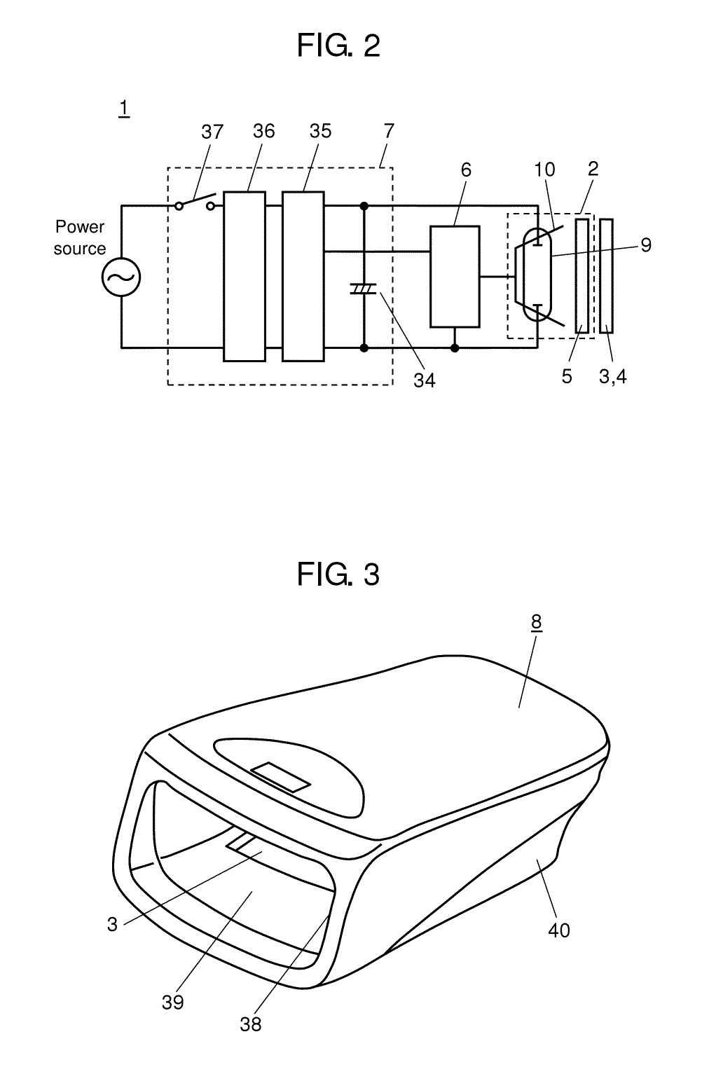

Hereinafter, a photoradiation device in a first exemplary embodiment of the present invention and a photoradiation therapy/prophylaxis device using the photoradiation device are described with reference to FIGS. 2 to 4.

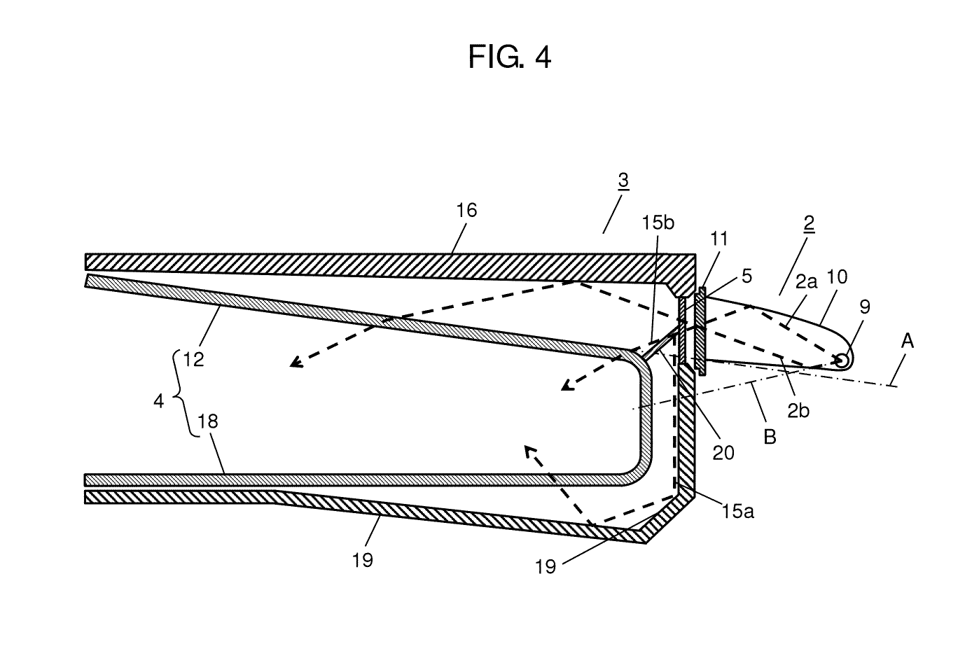

FIG. 2 is a control circuit diagram of the photoradiation therapy/prophylaxis device in accordance with the exemplary embodiment of the present invention. FIG. 3 is an entire perspective view of the photoradiation therapy/prophylaxis device in accordance with this exemplary embodiment. FIG. 4 is a sectional view of an optical system of the photoradiation therapy/prophylaxis device in accordance with this exemplary embodiment.

As photoradiation therapy/prophylaxis device 1 of this exemplary embodiment, a photoradiation device is described as an example, which is used for persons to be treated who undergo preventive treatment for preventing an affection of an inflammatory disease or reducing a symptom of the disease at the affection, or persons to be treated (patients) who undergo treatment of an inflammatory disease by inhibiting the inflammatory disease.

Firstly, as shown in FIGS. 2 to 4, photoradiation therapy/prophylaxis device 1 of this exemplary embodiment includes at least light emitting unit 2 which radiates radiation light, reflection unit 3, light guide unit 4, wavelength transmitting unit 5, light emission control unit 6, light source supply unit 7, and device main body 8 (see FIG. 3). Reflection unit 3 reflects radiation light radiated from light emitting unit 2 to light guide unit 4. Light guide unit 4 allows reflected light reflected by reflection unit 3 to transmit and to be guided to an irradiation target body. Wavelength transmitting unit 5 allows radiation light in a specific wavelength in radiation light radiated from light emitting unit 2 to transmit. Light emission control unit 6 controls light emission of light emitting unit 2, and light source supply unit 7 supplies light emitting unit 2 and light emission control unit 6 with electricity.

Note here that light emission control unit 6 controls light emission of light emitting unit 2 by the following light emission pattern. For example, light emission control unit 6 allows light emitting unit 2 to flash once or a plurality of times. At this time, when light emission control unit 6 allows light emitting unit 2 to flash a plurality of times, furthermore, it may allow light emitting unit 2 to flash with radiated radiating energy suppressed to not more than a predetermined radiating energy. Furthermore, light emitting unit 2 controls to emit light at predetermined light emitting intervals.

Furthermore, light source supply unit 7 shown in FIG. 2 includes storage section 34, charging circuit 35, light source unit 36, and light source switch 37 for turning on and off of light source unit 36. Note here that light source supply unit 7 is also used as a light source of light emission control unit 6.

Storage section 34 includes a main capacitor having electric capacitance necessary for allowing, for example, light emitting unit 2 to emit light and connected in parallel to light source 9, and stores light-emitting energy of light emitting unit 2. Charging circuit 35 charges storage section 34 with electricity supplied via light source unit 36. Light source unit 36 includes, for example, a plug which is connected to a plug receptacle (light source outlet) and receives supply of electricity and a light source cable, and supplies storage section 34 with electricity. Note here that light source unit 36 may include battery, a battery charger, or the like. Thus, portability of a photoradiation device is improved.

Furthermore, device main body 8 shown in FIG. 3 has a structure accommodating light emitting unit 2, reflection unit 3, light guide unit 4, wavelength transmitting unit 5, light emission control unit 6, and light source supply unit 7, and is capable of irradiating a region to be prevented or an affected region (a specific region) of a user with transmitted light (radiation light) transmitted from wavelength transmitting unit 5.

Then, device main body 8 is formed in, for example, a substantially rectangular parallelepiped shape (including a rectangular parallelepiped shape) having at least one opening, and has a casing incorporating light emitting unit 2, reflection unit 3, light guide unit 4, wavelength transmitting unit 5, light emission control unit 6, light source supply unit 7, and the like.

Furthermore, device main body 8 includes at least mount part 39, and grasping part 40 for grasping to carry device main body 8. Mount part 39 is a base on which a user inserts and puts a hand from opening part 38 formed on one face (hereinafter, which is referred to as a “front face”) of device main body 8 in order to irradiate, for example, a back of a hand with radiation light having a specific wavelength range.

Next, light emitting unit 2 accommodated in device main body 8 is described with reference to FIG. 4.

As shown in FIG. 4, light emitting unit 2 includes at least light source 9, reflector 10, and Fresnel lens 11. Fresnel lens 11 is installed on an opening part of reflector 10 and matches a light-entering angle of radiation light entering wavelength transmitting unit 5.

At this time, light emitting unit 2 is provided at an opposite side (upper side in the drawing) to an irradiation target body with respect to tangent line A (in this exemplary embodiment, a line on a face on which first light guide face 12 of light guide unit 4 that is an extended face).

Furthermore, light source 9 of light emitting unit 2 is formed of, for example, a (flash) discharge tube such as a xenon discharge tube and a halide discharge tube, and irradiates a region to be prevented or an affected region of a living body with radiation light in the wavelength which inhibits production of inflammatory cytokines. This exemplary embodiment describes an example in which a xenon discharge tube is used for light source 9.

Furthermore, reflector 10 of light emitting unit 2 reflects, for example, radiation light 2a, which travels to an opposite side to an irradiation target body side of light guide unit 4 with respect to tangent line A that is in contact with first light guide face 12 of light guide unit 4, to reflection unit 3 or light guide unit 4. Similarly, reflector 10 reflects, for example, radiation light 2b, which travels toward tangent line A, that is, toward irradiation target body side of light guide unit 4, to reflection unit 3 or light guide unit 4.

Furthermore, Fresnel lens 11 is provided when a filter having light-entering angle dependence is used in, for example, wavelength transmitting unit 5. At this time, Fresnel lens 11 is provided in such a manner that a light-entering angle entering from light source 9 is within a permissible light-entering angle of wavelength transmitting unit 5 to be used. Note here that Fresnel lens 11 may be omitted when, for example, a colored glass filter which does not have light-entering angle dependence is used for wavelength transmitting unit 5.

Furthermore, reflection unit 3 controls an irradiation range of radiation light which is radiated into substantially all directions (including all directions) from light source 9 so as to irradiate a region to be prevented or an affected region (a specific region) with radiation light that has transmitted through wavelength transmitting unit 5.

Then, reflection unit 3 of this exemplary embodiment includes first reflection unit 16 reflecting radiation light radiated from light emitting unit 2 to first light guide face 12 of light guide unit 4, third reflection unit 20 reflecting a part of the radiation light radiated from light emitting unit 2 to second reflection unit 19, and second reflection unit 19 reflecting radiation light reflected by third reflection unit 20 to second light guide face 18 that is different from first light guide face 12 of light guide unit 4.

At this time, as shown in FIG. 4, first reflection unit 16 of reflection unit 3 is disposed facing first light guide face 12 of light guide unit 4. First reflection unit 16 of reflection unit 3 and first light guide face 12 of light guide unit 4 are formed such that a space therebetween is narrower as a distance from light emitting unit 2 is increased. That is to say, first reflection unit 16 and first light guide face 12 are disposed such that an optical path length between a reflection face of first reflection unit 16 and first light guide face 12 of light guide unit 4 is shortened as a position at which the reflected light of light guide unit 4 is allowed to transmit is more distant from light emitting unit 2. Specifically, in this exemplary embodiment, as shown in FIG. 4, for example, first reflection unit 16 of reflection unit 3 is provided in a horizontal direction, and first light guide face 12 of light guide unit 4 is provided inclined such that a distance with respect to first reflection unit 16 of reflection unit 3 is shortened (narrowed) from a light emitting unit 2 side toward an opposite side (left side in the drawing) of light emitting unit 2. Note here that first reflection unit 16 is formed substantially continuously (including continuously) from reflector 10 of light emitting unit 2, and provided to a position of an end portion at the opposite side to light emitting unit 2 with respect to first light guide face 12.

On the other hand, second reflection unit 19 of reflection unit 3 is disposed opposite side facing first reflection unit 16 with first light guide face 12 and second light guide face 18 constituting light guide unit 4 sandwiched therebetween. Second reflection unit 19 is formed such that a space between second reflection unit 19 of reflection unit 3 and second light guide face 18 of light guide unit 4 becomes narrower as a distance from light emitting unit 2 is increased. That is to say, second reflection unit 19 and second light guide face 18 are arranged such that an optical path length between a reflection face of second reflection unit 19 and second light guide face 18 of light guide unit 4 is shortened as a position at which the reflected light of light guide unit 4 is allowed to transmit is more distant from light emitting unit 2. Specifically, in this exemplary embodiment, as shown in FIG. 4, for example, second light guide face 18 of light guide unit 4 is provided in a horizontal direction, and second reflection unit 19 of reflection unit 3 is provided inclined such that a distance with respect to second light guide face 18 of light guide unit 4 is shortened (narrowed) from a light emitting unit 2 side toward an opposite side (left side in the drawing) of light emitting unit 2. Note here that the inclination of second reflection unit 19 of reflection unit 3 is formed in the middle of second light guide face 18 of light guide unit 4, but it is needless to say that the inclination is not necessarily limited to this. The reason for the above mention is because the photoradiation device of this exemplary embodiment is configured with irradiation of front and rear surfaces of the hand with radiation light is considered. That is to say, since a palm side has less production of inflammatory cytokine, irradiation to the palm is not carried out.

Furthermore, third reflection unit 20 of reflection unit 3 is a reflection plate that reflects radiation light radiated from light source 9 of light emitting unit 2, and is disposed such that, for example, it bridges between light guide unit 4 and wavelength transmitting unit 5. Third reflection unit 20 is disposed inclined toward Fresnel lens 11 constituting light emitting unit 2 and wavelength transmitting unit 5 provided to reflection unit 3. That is to say, third reflection unit 20 is disposed such that a tip side at wavelength transmitting unit 5 side of third reflection unit 20 is inclined toward light emitting unit 2 with a predetermined angle, for example, 45° with respect to wavelength transmitting unit 5 around the axis perpendicular to the direction from light emitting unit 2 to first reflection unit 16.

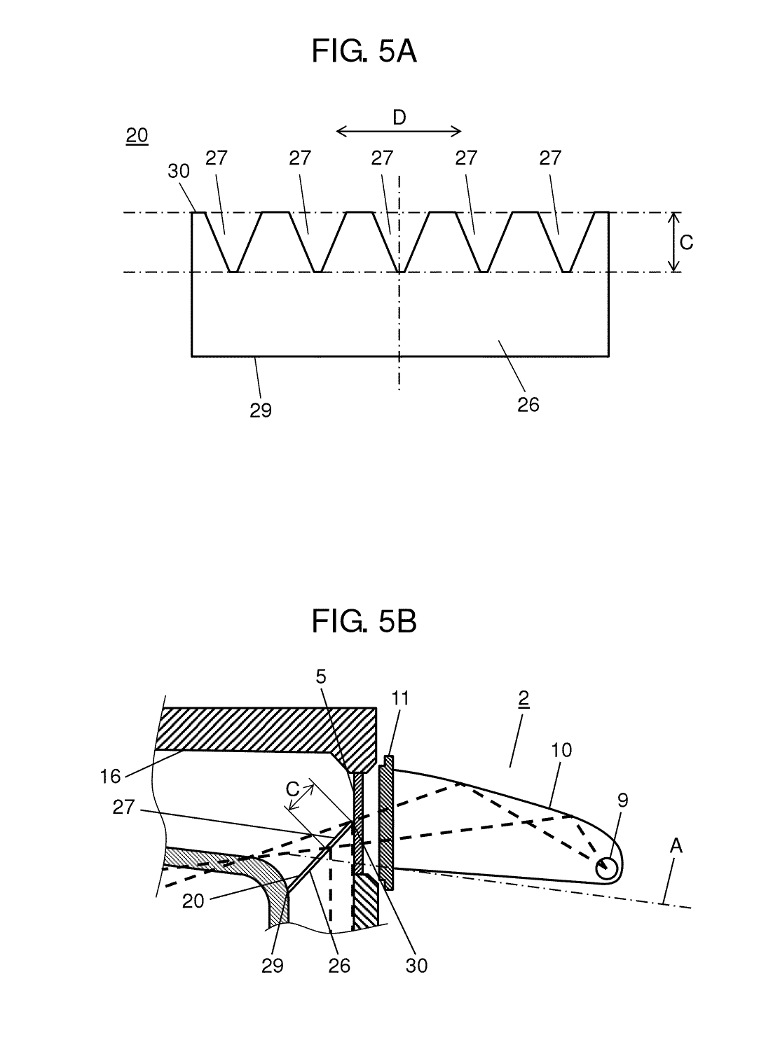

Hereinafter, a configuration and an effect of third reflection unit 20 are described in detail with reference to FIGS. 5A and 5B.

FIG. 5A is a front view showing a third reflection unit of the photoradiation therapy/prophylaxis device in accordance with the exemplary embodiment of the present invention. FIG. 5B is a partially enlarged sectional view for illustrating arrangement of the third reflection unit of the photoradiation therapy/prophylaxis device in accordance with this exemplary embodiment.

As shown in FIGS. 5A and 5B, third reflection unit 20 includes main body part 26 that reflects radiation light radiated from light emitting unit 2 to second reflection unit 19, and transmission unit 27 that allows a part of the radiation light radiated from light emitting unit 2 to transmit and forms an optical path to first reflection unit 16 by allowing.

Main body part 26 of third reflection unit 20 is formed in a flat shape, and base end part 29 of main body part 26 is fixed to first light guide face 12 of light guide unit 4. On the other hand, tip end part 30 of main body part 26 of third reflection unit 20 is extended to wavelength transmitting unit 5, and disposed inclined so as to cover a part of wavelength transmitting unit 5 (a half region of wavelength transmitting unit 5 in this exemplary embodiment). That is to say, a transmission unit side of the third reflection unit is disposed inclined toward a light emitting unit side. Thus, main body part 26 divides radiation light radiated from light emitting unit 2 into first reflection unit 16 and second reflection unit 19.

Furthermore, transmission unit 27 of third reflection unit 20 is provided in a region of position C from tip end part 30 of main body part 26 covering an optical path of the radiation light travelling toward a light emitting unit 2 side of first light guide face 12. At this time, a plurality of transmission units 27 are formed at, for example, a certain interval in wide direction D of main body part 26 (direction perpendicular to paper of FIG. 5B).

Furthermore, transmission unit 27 of third reflection unit 20 is formed in a plurality of portions of tip end part 30 of main body part 26 from tip end part 30 to base end part 29 in such a manner that it is notched in a shape of, for example, a triangular shape in this exemplary embodiment. At this time, transmission unit 27 of third reflection unit 20 is provided such that an opening area is extended as it is far away from first light guide face 12, that is, it is closer to light emitting unit 2. Thus, an amount of transmission light of the radiation light radiated from light emitting unit 2 to first reflection unit 16 is increased as transmission unit 27 is farer away from first light guide face 12, that is, it is closer to light emitting unit 2.

Furthermore, light guide unit 4 includes first light guide face 12 and second light guide face 18, which are disposed facing each other, as shown in FIG. 4. Note here that in this exemplary embodiment, first light guide face 12 is disposed facing the back of a hand (outside face from the wrist to the finger tip of the hand) of a user. On the other hand, second light guide face 18 is disposed facing the palm (inside face from the wrist to the finger tip of a hand) of a user.

Furthermore, wavelength transmitting unit 5 is disposed on optical path of radiation light radiated from light emitting unit 2 to first reflection unit 16 of reflection unit 3 and it is disposed at a light emitting unit 2 side from third reflection unit 20 of reflection unit 3 in this exemplary embodiment. Note here that wavelength transmitting unit 5 is formed of an optical filter through which radiation light of only one or more specific wavelength or only one or more specific wavelength ranges in radiation light from light emitting unit 2 transmits.

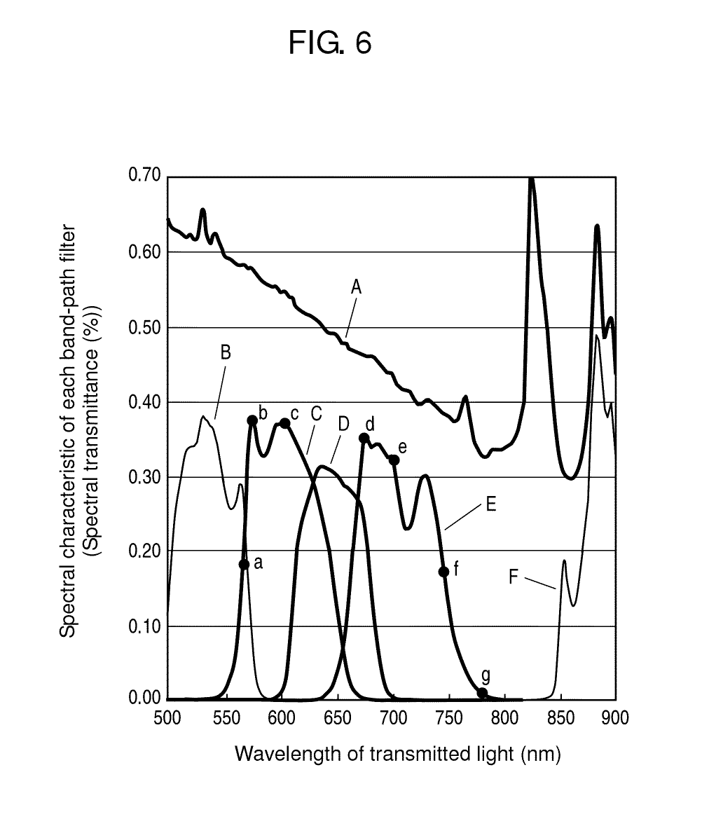

Hereinafter, as an optical filter of wavelength transmitting unit of this exemplary embodiment, effects and advantageous of wavelength transmitting unit 5 are described with reference to FIG. 6 taking a band-pass filter (interference filter) that selectively allows only radiation light in a specific wavelength range (wavelength band) to transmit is described as an example.

Specifically, wavelength transmitting unit 5 is a band-pass filter which allows radiation light in the wavelength range from of not less than 566.5 nm and not more than 780 nm to transmit.

FIG. 6 is a graph showing spectral characteristics of radiation light of a band-pass filter (wavelength transmitting unit) used in the photoradiation therapy/prophylaxis device in accordance with the exemplary embodiment of the present invention. Hereinafter, band-pass filters having spectral characteristics shown by solid lines C to E are referred to as band-pass filters C to E, respectively. Note here that solid line A of FIG. 6 shows spectral characteristic of the radiation light that does not transmit through the optical filter.

As shown in FIG. 6, firstly, a lower limit value of a wavelength range of the spectral characteristic (spectral transmittance) of the radiation light that has transmitted through each band-pass filter is a wavelength at a short wavelength side (wavelength corresponding to point “a” on solid line C) which is ½ (half) of the transmittance of the wavelength in which the transmittance of band-pass filter C having center wavelength of 600 nm shown by solid line C (wavelength corresponding to point “b” on solid line C: 566.5 nm). On the other hand, an upper limit value of the wavelength range of the spectral characteristic (spectral transmittance) of the radiation light that has transmitted through each band-pass filter is 780 nm, that is, a maximum wavelength of visible light.

That is to say, band-pass filter C having center wavelength of 600 nm of the radiation light is an optical filter that allows wavelength having an action of inhibiting a production amount of hVEGF as shown in FIG. 1A to transmit. Note here that band-pass filter B having spectral characteristic of center wavelength of 525 nm shown by solid line B in FIG. 6 at a shorter wavelength side than band-pass filter C has a lower effect for inhibiting the production amount of hVEGF is thought to be low from the results of FIG. 1A. Thus, it can be evaluated that wavelength at point “a” at the short wavelength side of band-pass filter C having center wavelength of 600 nm is a lower limit value having an action of inhibiting inflammatory cytokine.

On the other hand, since radiation light of near-infrared ray (wavelength of more than 780 nm) shown in FIG. 6 has a large thermal effect when irradiation is carried out to a user, the wavelength range to the near-infrared visible light (wavelength of not more than 780 nm) is defined as an upper limit value of the wavelength range at which wavelength transmitting unit 5 is allowed to transmit.

Note here that it is preferable that wavelength transmitting unit 5 is an optical filter that allows radiation light in a wavelength range of not less than 566.5 nm and not more than 746 nm to transmit. That is to say, the upper limit value of the wavelength range (not more than 746 nm) is a wavelength at a long-wavelength side (wavelength corresponding to point “f” of solid line E) that is ½ (half) of the transmittance in the wavelength (wavelength corresponding to point “d” on solid line E: 676.5 nm) in which the transmittance of band-pass filter E having spectral characteristic of the center wavelength of 700 nm shown by solid line E in FIG. 6 becomes maximum.

That is to say, band-pass filter E having center wavelength of 700 nm in the radiation light is an optical filter through which the wavelength, which has an action of inhibiting a production amount of hVEGF as shown in FIG. 1A, transmits. Note here that it is thought that band-pass filter F having spectral characteristic of center wavelength of 880 nm shown by solid line F in FIG. 6 at longer-wavelength side than band-pass filter E has less effect of inhibiting the production amount of hVEGF from the results of FIG. 1A. Thus, it can be evaluated that a wavelength at point “f” at a long-wavelength side of band-pass filter E of center wavelength 700 nm is a value having the action of inhibiting the inflammatory cytokine.

On the other hand, wavelength 780 nm that is an upper limit value of band-pass filter E (wavelength corresponding to point “g” on solid line E) is also at a shorter wavelength side than the wavelength of the radiation light that transmits through band-pass filter F having center wavelength of 880 nm. Therefore, the effect of inhibiting inflammatory cytokine by irradiation with radiation light of wavelength of 780 nm is not denied, and it can be evaluated that it has an inhibiting effect (operation).

Furthermore, as wavelength transmitting unit 5, an optical filter capable of allowing the radiation light within a wavelength range of not less than 600 nm and not more than 700 nm to transmit is more preferable.

That is to say, as shown in FIG. 1A, for example, band-pass filter C that is an optical filter having center wavelength of 600 nm (wavelength corresponding to point “c” on solid line C) which allows radiation light in the wavelength capable of inhibiting the production amount of hVEGF to transmit and band-pass filter E having center wavelength of 700 nm (wavelength corresponding to point “e” on solid line E) are used in combination. Thus, it is possible to inhibit the production of inflammatory cytokine at a high efficiency.

Furthermore, as shown in FIG. 1B, a wavelength range of radiation light which transmits through band-pass filter D having spectral characteristic of center wavelength of 650 nm shown by solid line D in FIG. 6, which has an effect of inhibiting the production of inflammatory cytokine, is located between band-pass filter C having center wavelength of 600 nm and band-pass filter E having center wavelength of 700 nm. That is to say, the wavelength ranges of band-pass filter C and band-pass filter E are overlapped with a wavelength range of band-pass filter D having spectral characteristic of center wavelength of 650 nm. Therefore, it is thought that radiation light having a wavelength which transmits through band-pass filter C having center wavelength of 600 nm and band-pass filter E having center wavelength of 700 nm has an effect of inhibiting production of inflammatory cytokine.

Hereinafter, an operation of photoradiation therapy/prophylaxis device 1 in accordance with an exemplary embodiment of the present invention is described specifically.

Firstly, a user inserts an irradiation target body, for example, a hand, from opening part 38 of device main body 8 shown in FIG. 3. At this time, the back of a hand of the user is defined as an upper side. That is to say, the user inserts the hand into device main body 8 in such a manner that the back of the hand faces a first light guide face 12 side and the palm faces a second light guide face 18 side as shown in FIG. 4. Note here that placement of the irradiation target body is not necessarily limited to the above-mentioned placement, it is needless to say that a user may insert the hand in such a manner that the back of the hand faces the second light guide face 18 side and the palm faces the first light guide face 12 side, that is to say, the rear surface and front surface are reversed.

Next, a light source switch (not shown) of device main body 8 is turned on in a state in which the irradiation target body, that is, the hand is inserted into device main body 8. Thus, light source of photoradiation therapy/prophylaxis device 1 is turned on and light emission control unit 6 emits light of light source 9 of light emitting unit 2 so as to radiate radiation light.

Then, radiation light radiated from light source 9 of light emitting unit 2 shown in FIG. 4 is directly radiated or indirectly radiated via reflection by reflector 10 toward Fresnel lens 11.

At this time, in radiation light that reaches Fresnel lens 11, a part of the radiation light, for example, radiation light 2b, which has transmitted through Fresnel lens 11 and is radiated to first light guide face 12 of light guide unit 4 or first reflection unit 16 transmits through first light guide face 12 and is irradiated to the back of a hand of a user.

On the other hand, remainder of radiation light that is other than the radiation light radiated from light emitting unit 2 and traveling toward first light guide face 12, for example, a part of radiation light 15a of radiation light 2a, is reflected by main body part 26 of third reflection unit 20, and guided to second reflection unit 19 as entering light. Then, radiation light 15a guided to second reflection unit 19 is reflected by second reflection unit 19 and irradiated to the palm of the hand of the user from second light guide face 18.

Usually, however, since radiation light entering first light guide face 12 of light guide unit 4 is blocked by third reflection unit 20, the light amount at a light emitting unit 2 side tends to be reduced. Then, in this exemplary embodiment, transmission unit 27 is provided to a tip end part 30 side of main body part 26 of third reflection unit 20 that is disposed inclined toward the light emitting unit 2 side. Thus, a part of radiation light 15b of radiation light 2a travelling toward third reflection unit 20 transmits through transmission unit 27 of third reflection unit 20, and a region at the light emitting unit 2 side of first light guide face 12 is irradiated. As a result, the amount of light at the light emitting unit 2 side of first light guide face 12 of light guide unit 4 can be increased.

Furthermore, as shown in FIG. 5A, transmission unit 27 of third reflection unit 20 disposed inclined toward light emitting unit 2 is formed in a shape whose opening area is increased at a tip end part 30 side that is closer to light emitting unit 2 in main body part 26. Therefore, a larger amount of the radiation light transmits through transmission unit 27 as a part separated from light emitting unit 2 of first light guide face 12 of light guide unit 4 (that is to say, a portion whose optical path length from light source 9 is increased). As a result, the illumination intensity distribution of first light guide face 12 of light guide unit 4 can be made to be uniform.

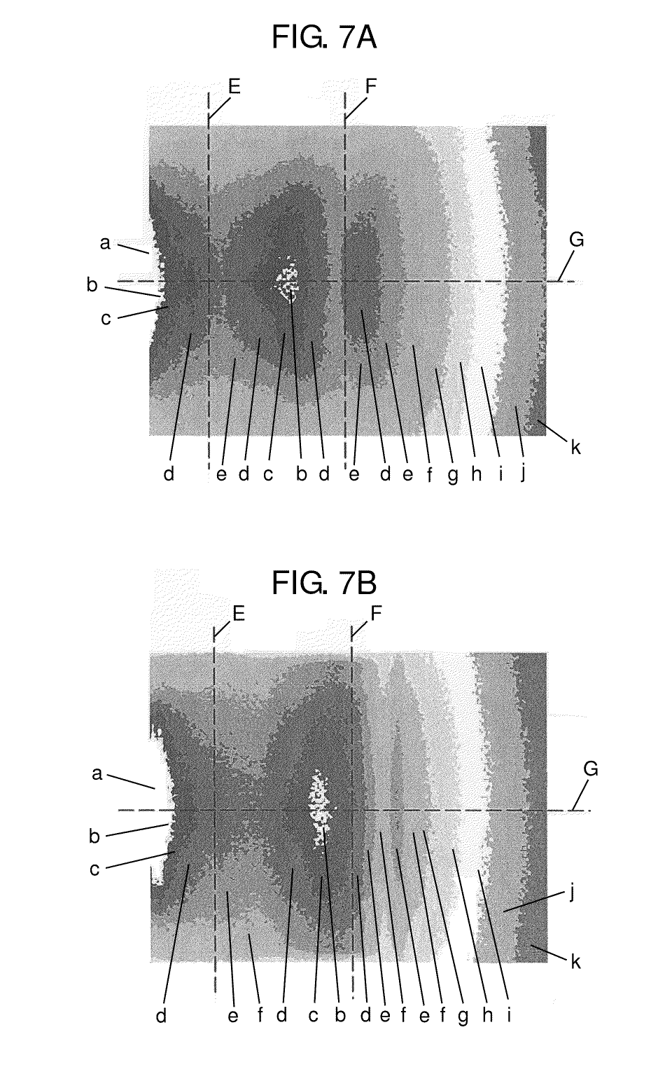

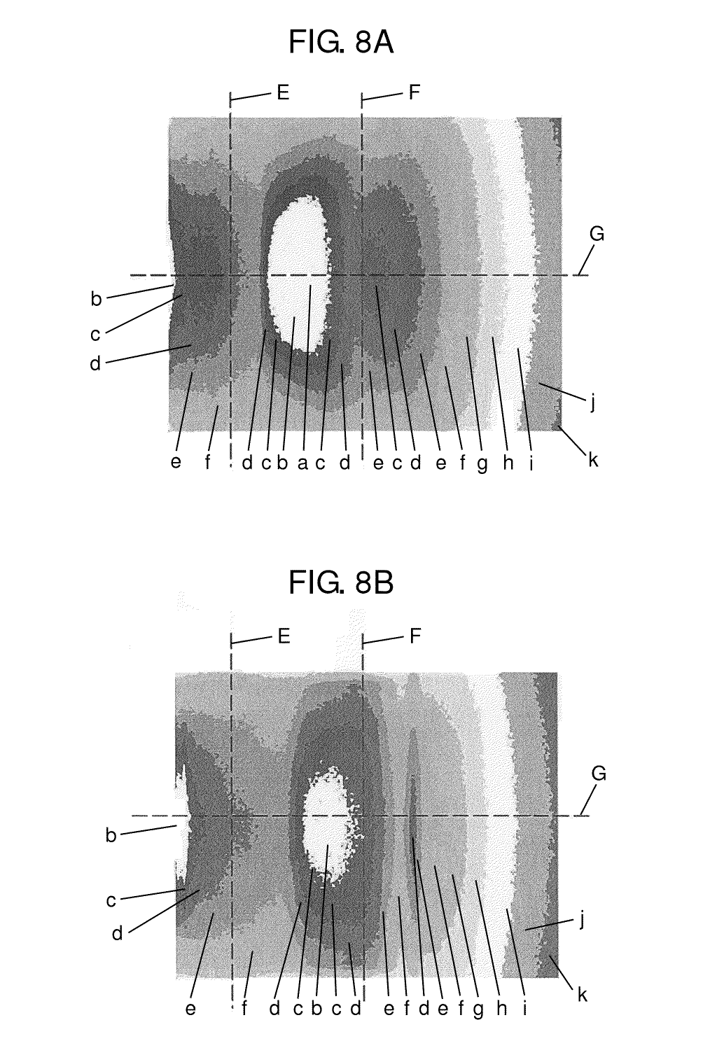

Hereinafter, effects and advantages of third reflection unit 20 having the above-mentioned configuration are specifically described with reference to FIGS. 7A to 8B.

FIG. 7A is a distribution diagram of illumination intensity in an irradiation target surface of radiation light irradiated from a first light guide face of the irradiation target surface of the photoradiation therapy/prophylaxis device in accordance with Example of this exemplary embodiment of the present invention. FIG. 7B is a distribution diagram of illumination intensity in an irradiation target surface of radiation light irradiated from a second light guide face of the irradiation target surface of the photoradiation therapy/prophylaxis device in accordance with Example of this exemplary embodiment. FIG. 8A is a distribution diagram of illumination intensity in an irradiation target surface of radiation light irradiated from a first light guide face of the irradiation target surface of the photoradiation therapy/prophylaxis device in accordance with a Comparative Example of the exemplary embodiment. FIG. 8B is a distribution diagram of illumination intensity in an irradiation target surface of radiation light irradiated from a second light guide face of the irradiation target surface of the photoradiation therapy/prophylaxis device in accordance with a Comparative Example of the exemplary embodiment.

Note here that FIG. 7A is a distribution diagram of illumination intensity in an irradiation target surface of radiation light irradiated to a back side of the hand from first light guide face 12, and FIG. 7B is a distribution diagram of illumination intensity in an irradiation target surface of radiation light irradiated to the palm side of the hand from second light guide face 18. Herein, the illumination intensity distribution diagrams of FIGS. 7A and 7B are shown such that the left end in the drawing corresponds to the position of an end portion at the light emitting unit 2 side of light guide unit 4. The direction from the left side to the right side in FIGS. 7A and 7B corresponds to a direction from the back surface side toward the front surface side (opening part 38 side) of device main body 8 of FIG. 3.

Furthermore, a position shown by dotted line E at an end portion side at a light emitting unit 2 side of first light guide face 12 shown in FIGS. 7A and 7B corresponds to a position at which the finger tip of a general user is placed (a position that is about 20 mm away from the end portion at the light emitting unit 2 side of light guide unit 4). On the other hand, a position shown by dotted line F corresponds to a position at which the position of the root of the finger of a general user is placed (a position that is about 100 mm away from the end portion at the light emitting unit 2 side of light guide unit 4).

Furthermore, reference marks a, b, c, d, e, f, g, h, i, j, and k shown in FIGS. 7A and 7B show predetermined regions (positions) of illumination intensity measured on the irradiation target surface. Specifically, reference mark “a” shows a region showing the illumination intensity of 6001× or more, and reference marks b to k show the illumination intensities at an interval of 6001× to 501× in descending order.

On the other hand, FIGS. 8A and 8B show illumination intensity distribution diagrams in the irradiation target surface when a third reflection unit (including only a main body part) without including transmission unit 27 is used as Comparative Examples for comparison with illumination intensity distribution of third reflection unit 20 including transmission unit 27 in this exemplary embodiment shown in FIGS. 7A and 7B. Note here that other configurations and reference marks, and the like, are the same as in FIGS. 7A and 7B, and therefore the description therefor is omitted.

Then, when Example of FIG. 7A showing the illumination intensity distribution of radiation light radiated from first light guide face 12 in the irradiation target surface and Comparative Example of FIG. 8A are compared with each other, the gradient of the illumination intensity distribution in Example is more gentle as compared with that of Comparative Example, showing that the illumination intensity is distributed uniformly.

Similarly, when Example of FIG. 7B showing the illumination intensity distribution of radiation light radiated from second light guide face 18 in the irradiation target surface and Comparative Example of FIG. 8B are compared with each other, the gradient of the illumination intensity distribution in Example is more gentle as compared with that of Comparative Example, showing that the illumination intensity is distributed uniformly.

That is to say, it is shown that when third reflection unit 20 is provided with transmission unit 27, the illumination intensity distribution in the irradiation target surface can be made to be uniform.

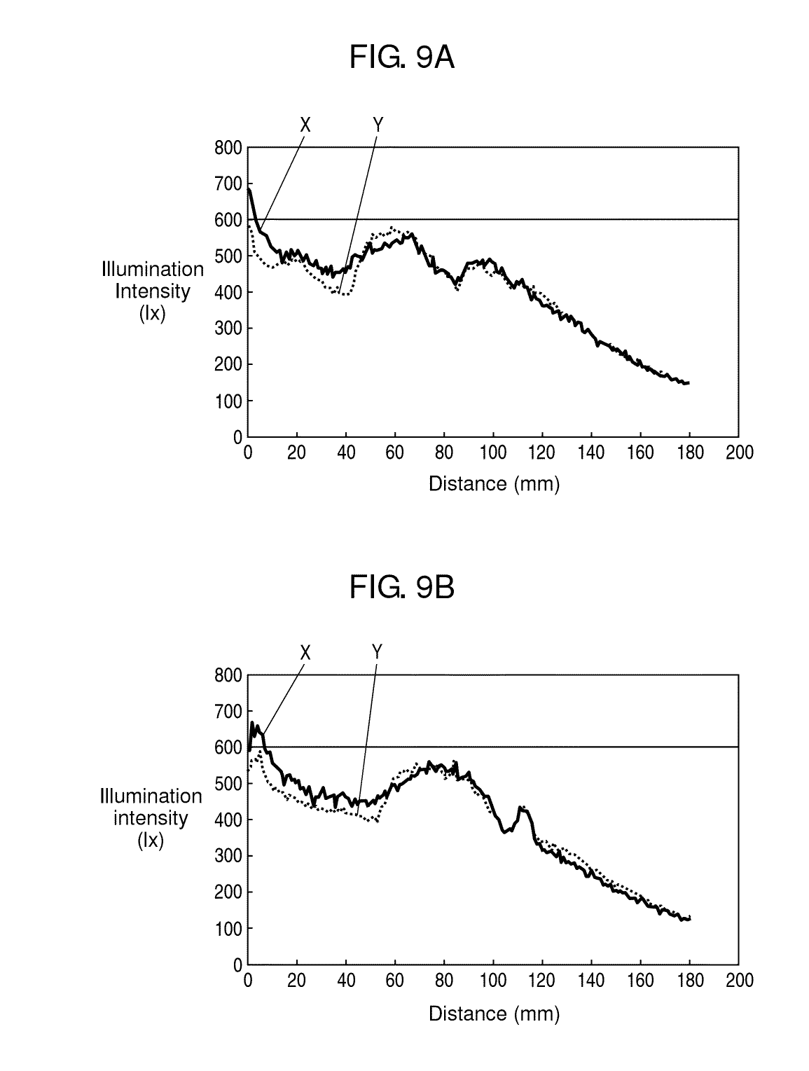

Hereinafter, the difference in the illumination intensity distribution in Example and Comparative Example in FIGS. 7A and 8A and FIGS. 7B and 8B is described with reference to FIGS. 9A and 9B in more detail.

FIG. 9A is a graph showing a relation between a position of an irradiation target surface of the radiation light irradiated from the first light guide face and illumination intensity in an illumination intensity distribution diagram in the photoradiation therapy/prophylaxis device in Example of FIG. 7A and Comparative Example of FIG. 8A. FIG. 9B is a graph showing a relation between a position of an irradiation target surface of radiation light irradiated from the second light guide face and illumination intensity in the illumination intensity distribution diagram of the photoradiation therapy/prophylaxis devices in Example of FIG. 7B and Comparative Example of FIG. 8B. Note here that abscissas of FIGS. 9A and 9B correspond to the positions shown by dotted line G of FIGS. 7A and 8A and FIGS. 7B and 8B.

Herein, solid lines X in FIGS. 9A and 9B show data of the illumination intensity distribution of this exemplary embodiment shown in FIGS. 7A and 7B; and broken lines Y show data of the illumination intensity distribution of Comparative Examples shown in FIGS. 8A and 8B.

When solid lines X and broken lines Y of FIGS. 9A and 9B are compared with each other, the gradient of the illumination intensity distribution in Example is more gentle as compared with that of Comparative Example. It can be evaluated that the illumination intensity is distributed uniformly.

As described above, according to this exemplary embodiment, when third reflection unit 20 is provided with transmission unit 27, it is possible to achieve a photoradiation device in which the illumination intensity distribution on an irradiation target surface is uniform and a photoradiation therapy/prophylaxis device having the photoradiation device.

Furthermore, according to this exemplary embodiment, light guide unit 4 has a plurality of light guide faces including first light guide face 12 and second light guide face 18. Then, to each of light guide faces of the light guide unit, a part of the radiation light radiated from light emitting unit 2 is guided to second reflection unit 19 indirectly via reflection by third reflection unit 20. Then, an irradiation target body can be irradiated from a plurality of the light guide faces of light guide unit 4 corresponding to the reflection units by reflecting the radiation light by any of first reflection unit 16 and second reflection unit 19 of reflection unit 3. As a result, it is possible to emit radiation light to a plurality of light guide faces by one light emitting unit 2, and to irradiate an irradiation target body having front and rear sides, for example, a hand.

Note here that the photoradiation therapy/prophylaxis device of the present invention is not necessarily limited to each of the above-mentioned exemplary embodiments, and it is needless to say that it can be modified variously in a scope that is not beyond the summary of the present invention.

For example, photoradiation therapy/prophylaxis device 1 in the above-mentioned exemplary embodiment is described as an example in which a hand is irradiated with radiation light, but photoradiation therapy/prophylaxis device 1 is not necessarily limited to this example. For example, other regions of a living body in which the production of inflammatory cytokine is to be inhibited or other affected regions of a living body may be irradiated. Furthermore, examples of regions of the living body include shoulder, hip, leg, whole body, and the like, and any regions may be irradiated. Furthermore, irradiation with radiation light is not necessarily limited to treatment of specific regions of a human body, and irradiation may be carried out for treatment of specific regions of non-human animals. In this case, it is needless to say that a structure is not limited to the structure of photoradiation therapy/prophylaxis device 1 in this exemplary embodiment and that the structure may be appropriately modified to a structure suitable for a specific region of a living body to be irradiated.

Furthermore, in photoradiation therapy/prophylaxis device 1 of this exemplary embodiment, a photoradiation device for irradiating regions of a living body in which production of inflammatory cytokine is to be inhibited or affected regions of a living body for the purpose of treatment and prevention is described as an example, but the photoradiation device is not limited to this example. The photoradiation device may be used as a lighting device necessary for efficiently and uniformly illuminating irradiation target bodies arranged or disposed uniformly, for example, general lighting, lighting of a sign board, lighting of a showcase of merchandises of a vending machine, backlight of liquid crystal display, in particular, a face lighting device. Thus, an irradiation target body can be uniformly irradiated with radiation light radiated from the light guide unit.

Furthermore, in photoradiation therapy/prophylaxis device 1 of this exemplary embodiment, an example provided with wavelength transmitting unit 5 through which radiation light in a specific wavelength range transmits is described, but photoradiation therapy/prophylaxis device 1 is not limited to the example. For example, when a photoradiation device is used for a face lighting device, wavelength transmitting unit 5 may not be provided. Furthermore, wavelength transmitting unit 5 that allows radiation light having a different wavelength (frequency) to transmit depending upon irradiation target bodies may be provided.

Furthermore, in photoradiation therapy/prophylaxis device 1 of this exemplary embodiment, wavelength transmitting unit 5 using a band-pass filter is described as an example, but it is not necessarily limited to this. For example, by combining a short-path filter (long-wavelength cut filter) and a long-path filter (short wavelength cut filter), wavelength transmitting unit 5 that selects to allow a specific wavelength range of entering light to transmit may be used.

Furthermore, in photoradiation therapy/prophylaxis device 1 of this exemplary embodiment, an example in which one or two cylindrical flash discharge tubes are used as light source 9 of light emitting unit 2 is described, but the light source is not limited to this example. For example, a light source may be two or more flash discharge tubes arranged in series in the axial direction or a plurality of linearly disposed LEDs. Furthermore, a light source corresponding to extension of with direction of the light guide unit may be used.

Furthermore, in photoradiation therapy/prophylaxis device 1 of this exemplary embodiment, light guide unit 4 having a flat light guide face is described as an example, light guide unit 4 is not limited to this example. For example, the light guide face of the light guide unit may be a circular arc shape that round toward the depth direction (a direction from an opening of the tip side of light guide unit 4 toward light emitting unit 2 side), or may be semicircular shape. In this case, it is preferable that the light emitting unit is disposed at an irradiation target body side from a tangent line that is brought into contact with an end portion at a light emitting unit side of the light guide face of the light guide unit.

Furthermore, in photoradiation therapy/prophylaxis device 1 of this exemplary embodiment, an example in which first light guide face 12 and second light guide face 18 of light guide unit 4 do not have a diffusion property is described, but photoradiation therapy/prophylaxis device 1 is not necessarily limited to this example. For example, a diffusion panel having a diffusion property is used for the light guide face of the light guide unit.

Furthermore, in photoradiation therapy/prophylaxis device 1 of this exemplary embodiment, an example in which one light emitting unit 2 has two faces, first light guide face 12 and second light guide face 18 of light guide unit 4, is described, but photoradiation therapy/prophylaxis device 1 is not necessarily limited to this example. For example, a configuration in which one light emitting unit 2 has three or more light guide faces may be employed. Thus, an irradiation target body having a plurality of faces, for example, not only two faces such as the front and rear surfaces of a hand but also side faces of a hand can be irradiated with radiation light radiated from one light emitting unit simultaneously. As a result, a photoradiation device capable of radiating radiation light to an irradiation target body for a short time with high efficiency and photoradiation therapy/prophylaxis device 1 including the photoradiation device can be achieved.

Furthermore, in photoradiation therapy/prophylaxis device 1 of this exemplary embodiment, a configuration which is notched in a triangular shape from tip end part 30 to base end part 29 side of main body part 26 of third reflection unit 20 is described as transmission unit 27, but the configuration is not necessarily limited to this.

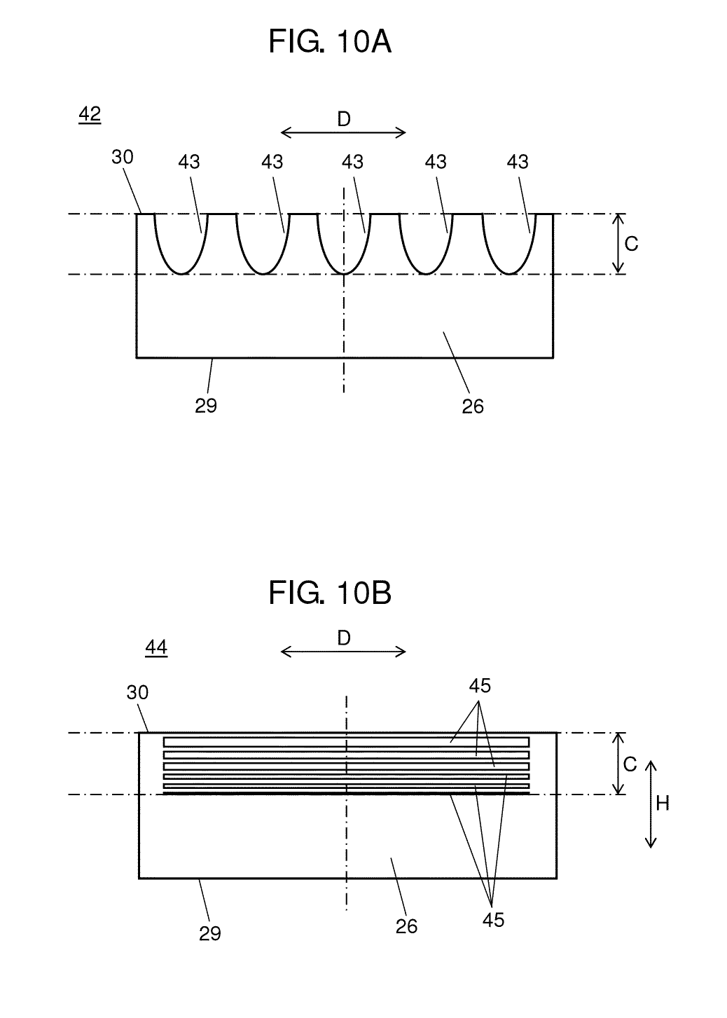

For example, as the transmission unit, a configuration described with reference to FIGS. 10A and 10B may be employed.

FIG. 10A is a front view showing a third reflection unit of another example of the photoradiation therapy/prophylaxis device in accordance with the exemplary embodiment of the present invention. FIG. 10B is a front view showing a third reflection unit of still another example of the photoradiation therapy/prophylaxis device in accordance with the exemplary embodiment of the present invention.

That is to say, as shown in FIG. 10A, transmission unit 43 of third reflection unit 42 may be notched in, for example, a semi-elliptical shape or a semicircular shape in a plurality of places from tip end part 30 to base end part 29 of main body part 26. At this time, transmission unit 43 of third reflection unit 42 is provided in such a manner that an opening area is increased as a distance from first light guide face 12 is increased, that is, it is closer to light emitting unit 2.

Furthermore, as shown in FIG. 10B, transmission unit 45 of third reflection unit 44 may be provided as a plurality of slits having a predetermined width in height direction H perpendicular to width direction D. At this time, slits as transmission units 45 are formed continuously from one end to the other end in width direction D of main body part 26 of third reflection unit 44. Then, the width of the slit at a tip end part 30 side of main body part 26 is formed smaller than the width of the slit at a base end part 29 side of main body part 26. Note here that when three or more slits are formed, it is preferable that the slits are formed such that the width of the slit is sequentially smaller from the tip end part 30 side to the base end part 29 side of main body part 26.

Thus, an amount of transmission light of radiation light radiated from light emitting unit 2 to first reflection unit 16 can be increased. Furthermore, the light amount at light emitting unit 2 side of first light guide face 12 of light guide unit 4 can be increased.

Furthermore, in photoradiation therapy/prophylaxis device 1 of this exemplary embodiment, a transmission angle of the radiation light that transmits through light guide unit 4 including first light guide face 12 and second light guide face 18 is not specifically described, but, for example, a small prism may be formed such that refraction is carried out with a smaller angle than the light-entering angle of light guide unit 4. Specifically, a plurality of small prisms are formed such that refraction is carried out with a smaller angle than the light-entering angle on a surface at an opposite side to the irradiation target body side of first light guide face 12 and second light guide face 18 (face that is not brought into contact with the irradiation target body). Thus, irradiation can be carried out such that the radiation light irradiated from light guide unit 4 is irradiated to the irradiation target body uniformly, and, simultaneously, the radiation light does not spread to the outside of the photoradiation device.

As described above, a photoradiation device of the present invention includes a light emitting unit that radiates radiation light; a reflection unit that reflects the radiation light; and a light guide unit that guides the radiation light reflected from the reflection unit to an irradiation target body. The light guide unit includes a first light guide face and a second light guide face. The reflection unit includes a first reflection unit that reflects a part of the radiation light to the first light guide face, a second reflection unit that reflects entering light to the second light guide face, and a third reflection unit formed of a main body part provided with a transmission unit which reflects a part of remainder of the radiation light to the second reflection unit as the entering light. Furthermore, the third reflection unit has a configuration in which a transmission unit side is disposed inclined toward a light emitting unit such that the shorter a distance of the transmission unit to the light emitting unit the larger an amount of transmission light of the radiation light.

With this configuration, a part of the radiation light radiated from the light emitting unit is reflected by the first reflection unit directly or via transmission of the transmission unit of the third reflection unit, or directly enters the first light guide face. Thereafter, the reflected radiation light also enters the first light guide face, and transmits through the first light guide face and is irradiated to one side of the irradiation target body. Furthermore, a part of the remainder of radiation light radiated from the light emitting unit is reflected by the third reflection unit, and reflected by the second reflection unit. Thereafter, the reflected radiation light enters the second light guide face, transmits through the second light guide face and is irradiated to the other side (opposite side) of the irradiation target body. That is to say, the photoradiation device of the present invention includes a plurality of the light guide faces to one light emitting unit.

Furthermore, the third reflection unit includes a main body part provided with a transmission unit, and the transmission unit side is disposed inclined toward the light emitting unit side. Thus, the radiation light radiated from the light emitting unit is uniformly distributed into the first light guide face and the second light guide face. Furthermore, the transmission unit of the third reflection unit is configured such that an amount of transmission light of the radiation light is increased as it is closer to the light emitting unit. Thus, the illumination intensity distribution in the first light guide face can be adjusted uniformly.

Furthermore, in the photoradiation device of the present invention, the third reflection unit is formed of a flat reflection plate. With this configuration, the third reflection unit can be formed in a simple configuration. Thus, high productivity and low cost can be achieved.

Furthermore, in the photoradiation device of the present invention, the transmission unit is a notch whose opening area increases with decrease in distance to the light emitting unit.

Furthermore, in the photoradiation device of the present invention, the transmission unit is formed in a slit whose opening area increases with decrease in distance to the light emitting unit.

With this configuration, the illumination intensity distribution on the first light guide face can be adjusted to be uniform.

Furthermore, the present invention relates to a photoradiation therapy/prophylaxis device which carries out treatment or prevention by irradiating a specific region of a living body with radiation light radiated from the above-mentioned photoradiation device. The present invention has a configuration further including a wavelength transmitting unit which is disposed on an optical path of the radiation light radiated from the light emitting unit through the light guide unit, and allows radiation light having a wavelength range of not less than 566.5 nm and not more than 780 nm in the radiation light radiated from the light emitting unit, and irradiating a specific region of a living body with radiation light that has transmitted through the wavelength transmitting unit.

With this configuration, the light guide unit includes a plurality of light guide faces. To each of the light guide faces of the light guide unit, a part of radiation light radiated from the light emitting unit is reflected by the third reflection unit to be indirectly guided to the second reflection unit. Then, the radiation light is reflected by any of the first reflection unit and the second reflection unit of the reflection unit. Thus, an irradiation target body can be irradiated from a plurality of the light guide faces of the light guide unit corresponding to each of the reflection units. As a result, it is possible to emit radiation light to a plurality of light guide faces by one light emitting unit 2, and to irradiate an irradiation target body having front and rear sides, for example, a hand.

Furthermore, with this configuration, it is possible to inhibit the production of inflammatory cytokine by irradiating regions in which various diseases (for example, inflammation, rough skin, and the like) are to be prevented or affected regions with radiation light that has transmitted through the wavelength transmitting unit. As a result, the production of inflammatory cytokine is inhibited to prevent affection of, for example, inflammatory disease, and to reduce or inhibit symptoms at the time of affection of the disease.

Specifically, the wavelength transmitting unit having the above-mentioned configuration allows the radiation light having wavelength of not less than 566.5 nm and not more than 780 nm, which has an action of inhibiting the production of inflammatory cytokine, which is discovered by the applicant of the present application, to transmit. At this time, wavelength of 780 nm that is an upper limit value, which has transmitted through the wavelength transmitting unit, is an upper limit value of the visible light (ray). Therefore, while the production of inflammatory cytokine can be inhibited, it is possible to minimize a thermal effect at the time of irradiation of regions in which an affection of disease is to be prevented or affected regions (including regions which are medically related to the regions, so that the treatment effect is shown).

### INDUSTRIAL APPLICABILITY

The present invention can be applied for a photoradiation device, a photoradiation therapy/prophylaxis device, and the like, in which radiation light radiated from a light source of the light emitting unit needs to be irradiated to a plurality of light guide faces of the light guide unit uniformly and simultaneously.

### REFERENCE MARKS IN THE DRAWINGS

1 photoradiation therapy/prophylaxis device
2 light emitting unit
2a, 2b, 15a, 15b radiation light
3 reflection unit
4 light guide unit
5 wavelength transmitting unit
6 light emission control unit
7 light source supply unit
8 device main body
9 light source
10 reflector
11 Fresnel lens
12 first light guide face
16 first reflection unit
18 second light guide face
19 second reflection unit
20, 42, 44 third reflection unit (reflection plate)
26 main body part
27, 43, 45 transmission unit
29 base end part
30 tip end part
34 storage section
35 charging circuit
36 light source unit
37 light source switch
38 opening part
39 mount part
40 grasping part

1. A photoradiation device comprising:
a light emitting unit that radiates radiation light;
a reflection unit that reflects the radiation light; and
a light guide unit that guides the radiation light reflected from the reflection unit to an irradiation target body,
wherein the light guide unit includes a first light guide face and a second light guide face that faces the first light guide face,
the reflection unit includes a first reflection unit that reflects a part of the radiation light to the first light guide face, a second reflection unit that reflects entering light to the second light guide face, and a third reflection unit formed of a main body part provided with a transmission unit which reflects a part of remainder of the radiation light to the second reflection unit as the entering light, and
the third reflection unit is disposed with a transmission unit side inclined toward a light emitting unit such that the shorter a distance of the transmission unit to the light emitting unit the larger an amount of transmission light of the radiation light.

2. The photoradiation device of claim 1, wherein the third reflection unit is formed of a flat reflection plate.

3. The photoradiation device of claim 1, wherein the transmission unit is a notch whose opening area increases with decrease in distance to the light emitting unit.

4. The photoradiation device of claim 1, wherein the transmission unit is a slit whose opening area increases with decrease in distance to the light emitting unit.

5. A photoradiation therapy/prophylaxis device for carrying out treatment or prevention by irradiating a specific site of a living body with radiation light radiated from a photoradiation device of claim 1, further comprising
a wavelength transmitting unit which is disposed on an optical path of the radiation light radiated from the light emitting unit through the light guide unit, and allows radiation light in a wavelength range of not less than 566.5 nm and not more than 780 nm in the radiation light radiated from the light emitting unit,
wherein a specific site of a living body is irradiated with the radiation light which is allowed to transmit through the wavelength transmitting unit.
